(19) United States
(12) Patent Application Publication
(10) Pub. No.: US 2014/0350463 A1
(43) Pub. Date: Nov. 27, 2014

(54) DUAL LUMEN PANCREATICOBILIARY CATHETER AND METHODS OF CANNULATING THE PANCREATICOBILIARY SYSTEM

(71) Applicant: BOSTON SCIENTIFIC SCIMED, INC., Maple Grove, MN (US)

(72) Inventors: Vanessa NAHON, Boston, MA (US); Lindsay MULLINS, Cambridge, MA (US); Steve WALAK, Natick, MA (US); Ralph BARRY, Hudson, MA (US)

(73) Assignee: BOSTON SCIENTIFIC SCIMED, INC., Maple Grove, MN (US)

(21) Appl. No.: 14/281,314

(22) Filed: May 19, 2014

Related U.S. Application Data
(60) Provisional application No. 61/826,403, filed on May 22, 2013.

(51) Int. Cl. A61M 25/01

(52) U.S. Cl. CPC A61M 25/0147; A61M 2025/015; A61M 25/09
USPC 604/95.04; 604/523; 604/528

(57) ABSTRACT

Devices and methods for facilitating access to the pancreaticobiliary system are disclosed. In particular, the methods include bending a device having an elongate body with a first lumen, a second lumen, and respective first and second exit openings. The methods include inserting a first guidewire through the first lumen and out of the first exit opening to cannulate a pancreatic duct, and inserting a second guidewire through the second lumen and out of the second exit opening to cannulate a biliary duct.

### CROSS-REFERENCE TO RELATED APPLICATIONS

This application claims the benefit of priority from U.S. Provisional Application No. 61/826,403, filed on May 22, 2013, the entirety of which is incorporated by reference herein.

### TECHNICAL FIELD

Various embodiments of the present disclosure relate generally to medical devices and related systems and methods. More specifically, the present disclosure relates to devices, systems, and methods for accessing the pancreaticobiliary system, e.g., to examine, diagnose, and/or treat a condition of the pancreatic duct and/or the bile duct.

### BACKGROUND

Access to the pancreaticobiliary system is required to diagnose and/or treat a variety of conditions, including tumors, gallstones, infection, sclerosis, and pseudocysts. One method of gaining access is via endoscopic retrograde cholangiopancreatography (ERCP), in which a side-viewing endoscope is passed down the esophagus, through the stomach, and into the duodenum where the duodenal papilla leading into the pancreatic and bile ducts may be visualized. In ERCP, tools such as sphincterotomes are passed through the working channel of the scope to gain access to the papilla, e.g., to investigate potential obstruction or inflammation of the pancreatic or bile ducts. Fluoroscopic contrast may be injected into either duct and X-ray images taken to determine the presence and location of strictures or stones.

Cannulation of either the bile duct or the pancreatic duct is a significant challenge in ERCP procedures. Factors that may complicate insertion into the papilla include sphincter orientation, floppy intraductal segments, biliary/pancreatic take-off levels, and the presence of stones or strictures. Difficult cannulations carry a high risk of perforation or other damage to tissue. For example, one technique physicians use to cannulate the papilla is to identify a bile trail, e.g., by pushing against the ampulla or applying suction to encourage bile from the duct. Prolonged probing, however, may lead to inflammation of the papilla and adverse effects for the patient.

Complications also may arise when the duct accessed first is not the duct desired for the procedure. When biliary access is desired, for example, a physician first may gain access to the pancreatic duct, e.g., via a guidewire. The physician then would have to remove the wire and attempt cannulation again. The pancreatic duct may be entered unintentionally several more times before access to the bile duct is finally achieved. These multiple pancreatic injections can irritate the tissue of the pancreatic duct and cause post-ERCP complications such as pancreatitis.

Thus, there remains a need for alternative methods of accessing the pancreaticobiliary system in order to improve efficacy of medical treatment and increase patient safety.

### SUMMARY OF THE DISCLOSURE

The present disclosure includes devices, systems, and methods for cannulating the pancreatic and biliary ducts such as during an ERCP procedure.

In accordance with an aspect of the present disclosure, a device for accessing anatomical openings in the pancreaticobiliary system of a patient may include an elongate body that may include a proximal end, a tapered distal end configured to bend so as to approach the anatomical openings, a first lumen extending between the proximal end and a first exit opening at the tapered distal end, and a second lumen extending from the proximal end to a second exit opening at the tapered distal end, in which the first and second exit openings are configured relative to each other so at the same time, each of the first and second exit openings faces a corresponding anatomical opening.

Various embodiments of the device may include one or more of the following features: the first exit opening may be configured to be oriented at about an eleven o'clock position and the second exit opening may be configured to be oriented at about a five o'clock position when the elongate body is viewed toward the distal end from the proximal end, the second lumen may be non-concentric with the first lumen, at least a portion of the tapered distal end may include an inclined guide component, the elongate body may have an actuation component configured to bend the tapered distal end to an orientation so as to approach the anatomical openings, the actuation component may be a wire attached to the tapered distal end, the second exit opening may be proximal to the first exit opening, and/or the device may include a handle having at least two insertion ports in which the elongate body is connected to the handle and, a first guidewire and a second guidewire for insertion into the first and second lumens respectively.

Another aspect of the present disclosure includes a method of accessing a biliary duct and a pancreatic duct of a patient, the method may include bending an elongate body having a first lumen, a second lumen, and respective first and second exit openings; inserting a first guidewire through the first lumen and out of the first exit opening to cannulate a pancreatic duct; and inserting a second guidewire through the second lumen and out of the second exit opening to cannulate a biliary duct.

Various embodiments of the method may include one or more of the following features: the elongate body may further include an elongate body that includes a proximal end, a tapered distal end configured to bend so as to approach the anatomical openings, the second exit may be proximal to the first exit, the second lumen may be non-concentric with the first lumen, at least a portion of the tapered distal end may have an inclined guide component, the elongate body may include an actuation component configured to bend the tapered distal end to an orientation so as to approach the biliary and pancreatic ducts, the second exit opening may be proximal to the first exit opening, a step of actuating an actuating component to bend the elongate body, the actuating component may be a wire, providing a pulling force on a proximal end of the wire to bend the elongate body, cutting tissue using the wire, rotating the elongate body prior to bending the elongate body, and straightening the papilla.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 shows anatomical features of the pancreaticobiliary system.

FIG. 2 shows an access device accessing the pancreatic and biliary ducts according to one embodiment of the present disclosure.

FIG. 3 is a side view of an access device according to an embodiment of the present disclosure.

FIGS. 4A-4D show cross-sectional views of access devices according to embodiments of the present disclosure.

FIGS. 5A-5D show side views of a distal portion of the access device according to embodiments of the present disclosure.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts. The term “distal” refers to the end farthest away from a medical professional when introducing a device in a patient. By contrast, “proximal” refers to the end closest to the medical professional when placing a device in the patient.

### Overview

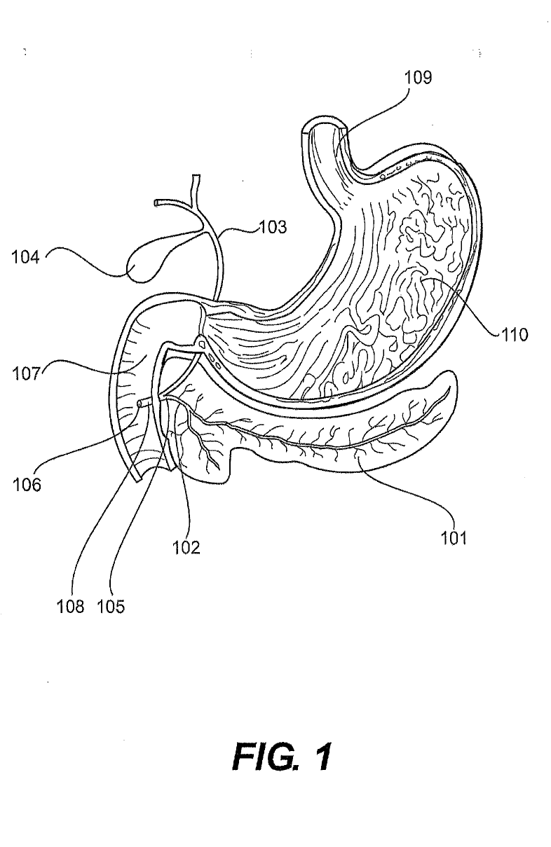

The pancreaticobiliary system, illustrated in FIG. 1, includes the pancreas (101), the pancreatic duct (102), the common bile or biliary duct (103), and the gallbladder (104). The pancreatic and biliary ducts join at the hepatopancreatic ampulla (105) (also known as the ampulla of Vader), which lies just behind the papilla (106). The papilla is a small opening that leads into the duodenum (107) to allow for the release of pancreatic juice and bile into the duodenum to aid in digestion. Smooth muscle of the hepatopancreatic sphincter (108) (also known as the sphincter of Oddi) regulates flow of pancreatic juice and bile into the duodenum.

Embodiments of the present disclosure relate to devices, systems, and methods for accessing portions of the pancreaticobiliary system of a patient. The device may have a flexible tip that may be adjusted in a variety of angles. The tip may be tapered to better facilitate cannulation. The device may have multiple lumens through which multiple devices may be inserted.

### Exemplary Embodiments

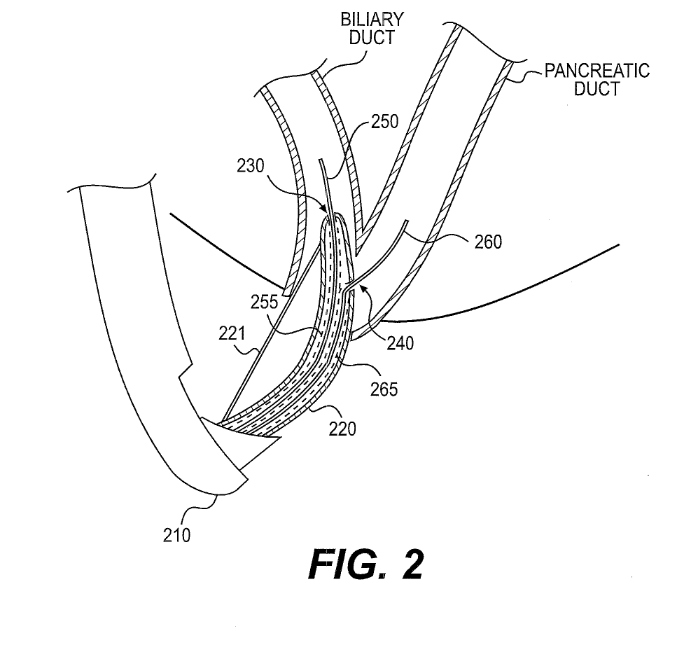

According to an embodiment of the present disclosure, as shown in FIG. 2, an elongated surgical device, such as a catheter (220), for example, a sphinctertome, may be inserted through a surgical device, such as an endoscope (210) to access an area of interest, for example, areas within the pancreatic duct or bile duct. The catheter (220) may include one or more lumens (255), (265) along at least a portion of its length.

A first surgical tool, such as a guidewire (250) may be inserted through a first lumen (255) of the catheter (220), out of the tapered distal end of the catheter (220) via exit opening (230) and into a portion of the body, for example, the biliary duct (as shown) or pancreatic duct. A second surgical tool, such as a guidewire (260) may be inserted through a second lumen (265) of the catheter (220) and may exit out of opening (240) of the catheter (220) and into an area of interest, for example, either the pancreatic duct (as shown) or the biliary duct.

The catheter (220) may have an open proximal end and a distal end and be formed of any material suitable for insertion in the body, for example, PTFE, suitable polymer, or other materials permitting flexibility to provide catheter (220) with ability to traverse tortuous anatomy. The catheter (220) may have a uniform or varying flexibility along its length and/or around the circumference. For example, the distal end of the catheter (220) may be more flexible than other parts of the catheter. The catheter (220) may include a coating having, for example, lubricious, and/or therapeutic properties, (e.g. antibiotic, anti-inflammatory, analgesic). In addition, the catheter (220) may be radiopaque and may include markings to allow visualization under x-ray or fluoroscopy.

The catheter (220) may include at least two lumens (255) and (265) along at least a portion of the length of the catheter (220) for passage of surgical tools, or delivery of therapeutic or diagnostic agents. For example, each lumen (255) and (265) may allow passage of a guidewire (250) and (260). The lumens (255) and (265) may have the same or different diameters. The space between the two lumens (255) and (265) may be varied along the catheter (220). The catheter (220) may include one or more additional lumens for the insertion of additional medical instruments and/or injection of fluids or gels such as, for example, contrast agent, and a hydrogel. Each lumen (255) and (265) may terminate on a distal portion of the catheter (220) in a respective exit opening (230) and (240).

The exit openings (230) and (240) may be holes or other opening and each may have a size suitable for allowing a guidewire (250) and (260) to pass through. The catheter (220) may be of sufficient length to allow passage through a surgical device, such as an endoscope (210). Endoscope (210) may include an elevator mechanism, or a ramp-like feature, to permit catheter (220) to exit a side of the distal tip of the endoscope, as shown in FIG. 2.

The catheter (220) may have a tapered portion, for example, the tapered portion may be on a distal end of the catheter (220). The tapered portion of the catheter (220) may have any size suitable for insertion in the body, for example, the tapered portion may have a size between about 3.5 to about 5.5 Fr.

The catheter (220) may include a component to control the movement, orientation, and/or curvature of the catheter (220), and particularly to control the catheter so that it approaches the ampulla (105) with the distal openings of the catheter facing or otherwise oriented towards the ducts. For instance, the distal end of the catheter (220) may be configured to curve in such a way as to approach the biliary and pancreatic ducts. For example, the catheter (220) may include a wire (221), electro-active polymer (EAP), electromagnetic component, or any other suitable actuating component to allow movement of the catheter (220).

For instance, the catheter (220) may include one or more wires (221) having a distal portion connected to the distal end of the catheter (220) and a proximal end controllable by an operator. The wire (221) may by actuate and controlled by an operator to control movement of the catheter (220). For example, the operator may either directly or indirectly exert an actuating force (e.g. by pulling, pushing, rotating or other suitable manner) on the proximal end of the wire (221), so as to actuate and control movement of the catheter (220), for example, to bow, bend, curve, or deflect the orientation of a portion of the catheter (220). As shown in FIG. 2, a distal end of wire (221) may be connected to a distal end of catheter (220) and may change the shape of the catheter (220) to a curved shape.

In another example, the catheter (220) may be formed in a pre-formed shape suitable for accessing a particular portion of the body. For instance, the catheter (220) may be manufactured to have one or more portions to have a pre-formed shape, such as a pre-curved distal end suitable for insertion into the pancreaticobiliary system.

In another example, the catheter (220) may be manufactured with a shape-memory material, such as nitinol. The shape-memory material may be configured to form a suitable shape upon insertion at the desired location. For example, a distal end of the catheter (220) may be manufactured with nitinol and may form a curved shape upon insertion into the body.

The catheter (220) may have any suitable shape, thickness, geometry, and/or orientation suitable for accessing a region of the body, which may be uniform or may vary along the length and/or around the catheter (220). For example, the distal end of the catheter (220) may be curved such that the exit openings (230) and (240) may be oriented at a predetermined desired orientation relative to each other. In this manner, the insertion tool, such as the guidewires (250) and (260) may exit the respective exit openings (230) and (240) at orientations suitable for entering the desired portion of the body. For example, openings (230) and (240) may be oriented relative to each other in any suitable orientation so as to allow access to the anatomical orifices of interest. These relative orientations may be based on the relative orientations of the anatomical orifices of interest and may be dependent on the specific anatomy of a patient.

For example, as shown in FIG. 2, the distal end of catheter (220) may have a curved shape configured to approach the anatomical orifices of interest, and exit openings (230) and (240) of respective lumens (255) and (265) may be oriented opposite to and about 180° relative to each other such that when viewed in cross-section from a proximal end, exit opening (230) may be at about an eleven o'clock position and exit opening (240) may be at about a five o'clock position, corresponding to the relative positions of the biliary and pancreatic ducts. In this manner, the curvature of the distal end of the catheter (220) and the orientation of the exit openings (230) and (240) may achieve effective access to the biliary duct and the pancreatic duct respectively.

The catheter (220) may include a tool for performing or aiding in the performance of a surgical procedure. For example, wire (221) may not only be configured to actuate and control movement of portions of the catheter (220), such as bend the distal end of the catheter (220), as described above, it may be manufactured from stainless steel or other suitable materials and may be configured to transmit high frequency electrical current to incise or cauterize a tissue, such as sphincter tissue. In another example, the catheter (220) may include a gripping tool for gripping tissue in the body, or any other tool.

The catheter (220) or endoscope (210) may include or be connected to a visualization component configured to visualize the catheter (220), devices placed through the catheter (220), such as guidewires (250) and (260) and/or the anatomical orifices of interest, such as the biliary duct and pancreatic duct and other parts of the pancreaticobiliary system. The visualization component may include, a scope component having a camera, fluoroscopic properties, or any other suitable visualization means. The visualization component may be used by the operator to orient the catheter (220) and/or the guidewires (250) and (260). For example, to properly orient the catheter (220) by visualizing movement of the movement component, such as wire (221).

Guidewires (250) and (260) may have the same or different diameters, which may be of any size suitable for insertion into the body, for example, for insertion into the pancreatic duct or biliary duct. For example, the guidewires (250) and (260) may have diameters ranging from about 0.018″ to about 0.035″. One or both of the guidewires (250) and (260) may have a solid metallic core with an applied coating. The coating may have, for example, lubricious, and/or therapeutic properties, and may be porous. The coating may have be radiopaque and include markings for visual indicators.

The guidewires (250) and (260) may have a constant flexibility along their lengths, or some portions of the guidewire may be more flexible than other portions. For example, the tips of the guidewires (250, 260) may be tapered and constructed of a softer material than other portions of the guidewires (250, 260) to promote cannulation of the papilla and minimize trauma. The guidewires (250, 260) may have a sufficient length to allow passage through the working channel of the endoscope (210) and allow the exchange of a catheter (220) over the guidewires (250, 260).

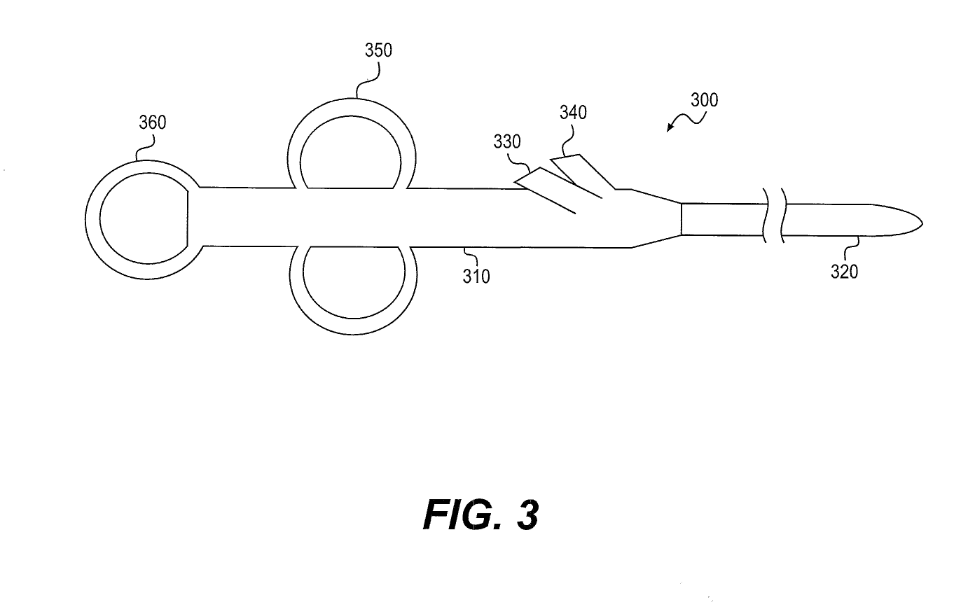

As shown in another embodiment in FIG. 3, device (300) may include a catheter (320) of the type discussed above, which may be connected to a handle portion (310) having two ports (330) and (340) through which a surgical device, fluid or other object may be inserted. For example, the ports (330) and (340) may have a diameter suitable for the insertion of guidewires. The handle portion (310) may be manufactured of any material having a sufficient rigidity to allow it to be gripped by an operator. For example, the handle portion may be manufactured by molding a plastic material. Each port (330) and (340) may include a covering or seal to prevent any contaminants from entering through the ports (330) and (340). The ports (330) and (340) may be disposed on the handle (310) at any suitable angle that may allow the operator to insert a device, such as a guidewire. The ports (330) and (340) may include distinguishing markings or have different colors to indicate which device/fluid/object should be inserted into which port (330), (340).

The handle (310) may include a thumbhole or grip (360) and finger-rings or actuators (350) for controlling, e.g., bending at the distal end through a wire (like wire 221) of the FIG. 2 embodiment. Handle (310) also may have a suitable electrocautery current from a generator to a wire (like wire 221). The grip (360) and actuators (350) may take any shape or geometry suitable for the operator to use. The handle (310) may remain outside of the body and in the operator's control.

In use, for example, if the desired procedure requires access to a first duct, such as the biliary duct, a first guidewire may be inserted through a first port of the handle into a first lumen of the catheter, out through a first exit opening of the catheter to access the second duct, such as the pancreatic duct. While the first guidewire is in place, a second guidewire may be inserted through another port of the handle into a different lumen of the catheter, out through a different exit opening of the catheter to access the first duct, such as the biliary duct. With the first guidewire already in the second duct, such as the pancreatic duct, the second duct, such as the pancreatic duct, may be essentially blocked and thus, access to the first duct, such as the biliary duct, may be more easily obtained.

With one guidewire in the second duct, such as the pancreatic duct, the disclosed procedure may also have the benefits of the double wire technique such as an anchoring of the device, and a straightening of the papilla. Utilizing this disclosed double wire technique may avoid multiple manipulations of the papilla and multiple contrast injections, which may lead to post-ERCP complications. The additional lumen could also be used to inject contrast into the system.

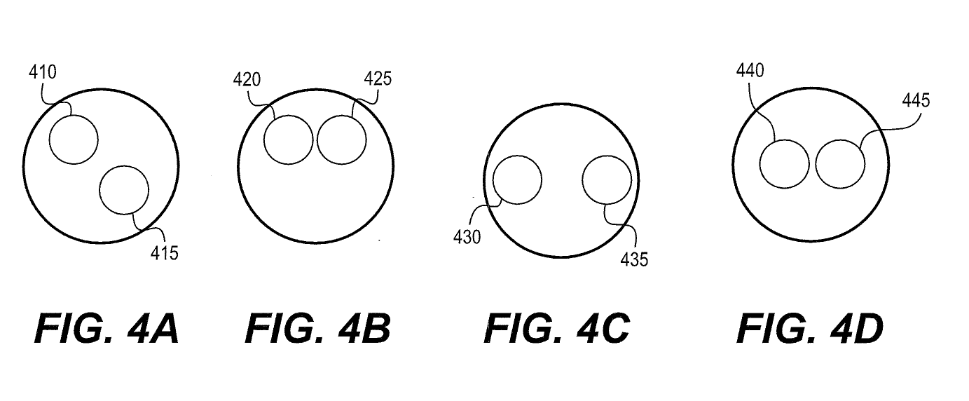

FIGS. 4A, 4B, 4C, and 4D show cross-sectional views of catheters according to embodiments of the disclosure, as viewed looking toward the distal end. The catheter may include dual lumens, with each lumen of the same or of a different size placed in a suitable position relative to the other. For example, when viewed looking toward the distal end from the proximal end, one lumen may be above the other lumen, or the two lumens may be horizontally aligned and parallel to each other. In other embodiments, when viewed looking toward the distal end from the proximal end, the lumens may be spaced about 180° apart, or any other suitable rotational relative position, and/or very close or as far apart as possible from each other, and/or any suitable orientation to access multiple orifices.

In one exemplary embodiment, as shown in FIG. 4A as a cross-sectional view of the catheter as viewed looking toward the distal end from the proximal end, lumen (410) may be at about an eleven o'clock position and lumen (415) may be at about a five to six o'clock position so that one or more of the inserted devices, such as a guidewire, may be oriented at a particular angle of approach when exiting the catheter tip. In this embodiment, lumens (410) and (425) are about 180° apart.

In another exemplary embodiment, as shown in FIG. 4B as a cross-sectional view of the catheter as viewed looking toward the distal end of the catheter from the proximal end, lumen (420) may be adjacent (very close to) lumen (425) and both lumens (420) and (425) may be above a center line of the catheter.

In another exemplary embodiment, as shown in FIG. 4C as a cross-sectional view of the catheter as viewed from the proximal end, lumen (430) may be spaced apart from lumen (435) and both lumens (430) and (435) may be aligned with the center line of the catheter and adjacent to the edge of the catheter (e.g. the two lumens may be as part apart as possible from each other). In another exemplary embodiment, as shown in FIG. 4D as a cross-sectional view of the catheter as viewed from the proximal end, lumen (440) may be adjacent to (very close to) lumen (445) and both lumens (440) and (445) may be aligned with the center line of the catheter.

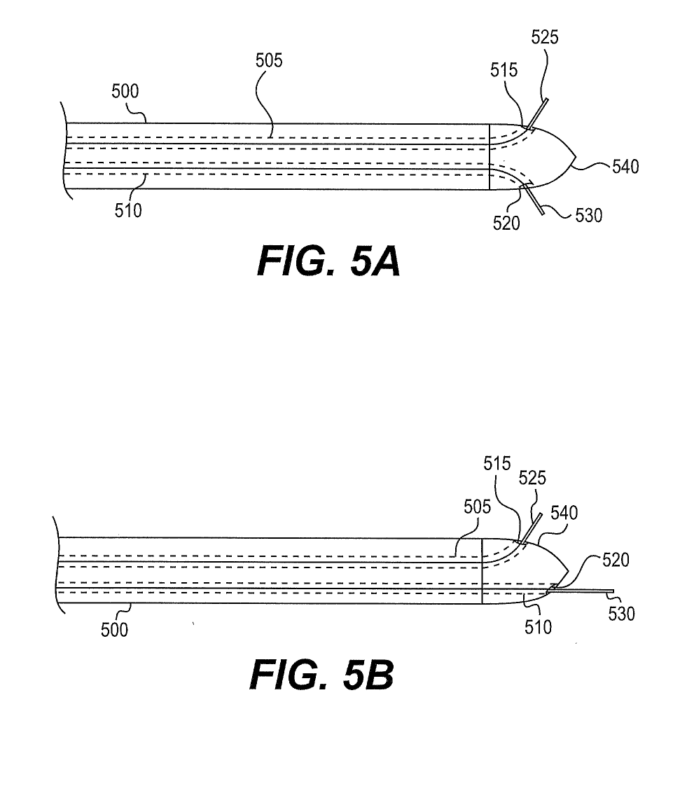

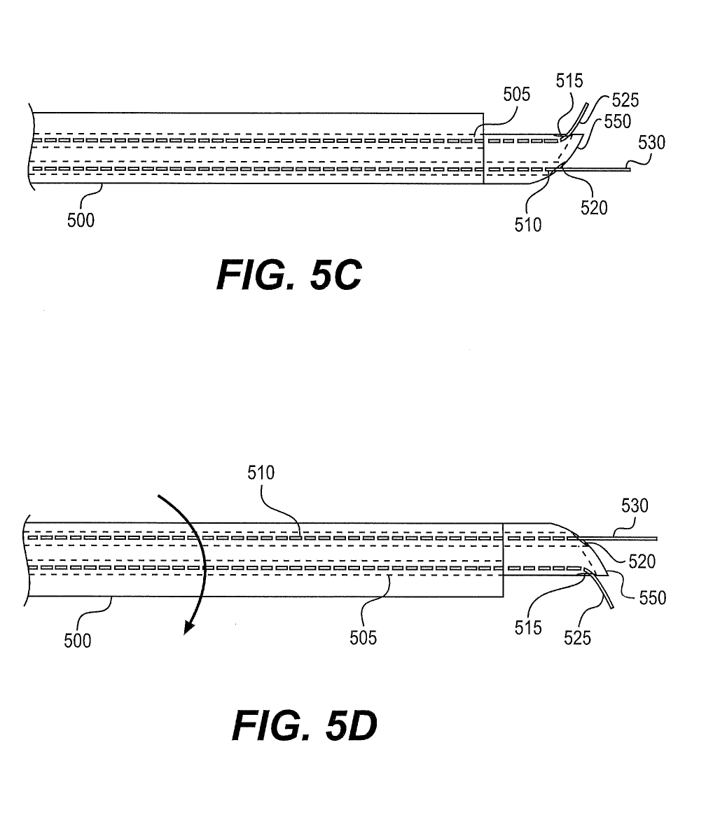

As shown in FIGS. 5A-5D, the catheter (500) may include a first lumen (505) for a first device, e.g. guidewire (525). The first lumen (505) may terminate at exit opening (515) at the tapered distal end (540) of the catheter (500). The catheter (500) may also include a second lumen (510) for a second device, e.g. guidewire (530). The second lumen (510) may terminate at exit opening (520) at the tapered distal tip (540) of the catheter (500).

FIG. 5A shows an exemplary embodiment in which the first and second exit opening (515) and (520) may be parallel to each other. The tapered distal end (540) of the catheter (500) may be configured such that the first and second guidewires (525) and (530) may exit via each of the respective exit openings (515) and (520) in an orientation suitable for accessing the anatomical sites of interest, such as the biliary duct and pancreatic duct. Furthermore, a suitable orientation for the guidewires (525) and (530) to access anatomical sites of interest may also be based on the curved or bowed orientation of the distal portion of the catheter (500) as discussed above.

In another example, the opening may be non-parallel relative to each other. For example, the first exit opening (515) may be either proximal or distal to the second exit opening (520). For example, as shown in FIG. 5B, the first exit hole (515) may be proximal to the second exit hole (510). In this example, as shown in FIG. 5B, guidewire (525) may be directed in an upwards orientation out of exit opening (515) and guidewire (530) may be directed in a horizontal direction out of exit opening (520). This configuration, as shown in FIG. 5B, is similar to the configuration shown in the cross-sectional view of a catheter when viewing the distal end of the catheter from a proximal end as shown in FIG. 4A and corresponds to the general anatomical spacing, direction, and orientation of the biliary and pancreatic ducts.

As shown in FIG. 5C, a ramp (550), cap, or deflector may be present at an end of the catheter (500) and may be used to deflect the guidewire (525) in lumen (505) out of the catheter (500) at any suitable angle via exit opening (515). For example, in FIG. 5C, guidewire (525) in lumen (505) may be directed upwards out of exit opening (515) and guidewire (530) in lumen (510) may be directed in a horizontal direction out of exit (520). As shown in FIG. 5D, the catheter (500) may be rotated, for instance about 180° , or any other suitable angle, so that guidewire (525) is directed downwards out of exit opening (515).

Embodiments of the present disclosure may be used in any medical or non-medical procedure, including any medical procedure where access to the pancreaticobiliary system is desired. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

We claim:

1. A device for accessing anatomical openings in the pancreaticobiliary system of a patient, the device comprising:
an elongate body that includes a proximal end, a tapered distal end configured to bend so as to approach the anatomical openings, a first lumen extending between the proximal end and a first exit opening at the tapered distal end, and a second lumen extending from the proximal end to a second exit opening at the tapered distal end,
wherein the first and second exit openings are configured relative to each other so at the same time, each of the first and second exit openings faces a corresponding anatomical opening.

2. The device of claim 1, wherein the first exit opening is configured to be oriented at about an eleven o'clock position and the second exit opening is configured to be oriented at about a five o'clock position when the elongate body is viewed toward the distal end from the proximal end.

3. The device of claim 1, wherein the second lumen is non-concentric with the first lumen.

4. The device of claim 1, wherein at least a portion of the tapered distal end comprises an inclined guide component.

5. The device of claim 1, wherein the elongate body comprises an actuation component configured to bend the tapered distal end to an orientation so as to approach the anatomical openings.

6. The device of claim 5, wherein the actuation component is a wire attached to the tapered distal end.

7. The device of claim 1, wherein the second exit opening is proximal to the first exit opening.

8. The device of claim 1, further comprising:
a handle having at least two insertion ports, wherein the elongate body is connected to the handle; and
a first guidewire and a second guidewire for insertion into the first and second lumens respectively

9. A method of accessing a biliary duct and a pancreatic duct of a patient, the method comprising:
bending an elongate body having a first lumen, a second lumen, and respective first and second exit openings;
inserting a first guidewire through the first lumen and out of the first exit opening to cannulate a pancreatic duct; and
inserting a second guidewire through the second lumen and out of the second exit opening to cannulate a biliary duct.

10. The method of claim 9, wherein the elongate body further comprises a proximal end, a tapered distal end configured to bend so as to approach the anatomical openings, wherein the second exit opening is proximal to the first exit opening.

11. The method of claim 10, wherein the second lumen is non-concentric with the first lumen.

12. The method of claim 10, wherein at least a portion of the tapered distal end comprises an inclined guide component.

13. The method of claim 10, wherein the elongate body comprises an actuation component configured to bend the tapered distal end to an orientation so as to approach the biliary and pancreatic ducts.

14. The method of claim 10, wherein the second exit opening is proximal to the first exit opening.

15. The method of claim 9, further comprising actuating an actuating component to bend the elongate body.

16. The method of claim 15, wherein the actuating component is a wire.

17. The method of claim 16, further comprising providing a pulling force on a proximal end of the wire to bend the elongate body.

18. The method of claim 9, further comprising a step of cutting tissue using the wire.

19. The method of claim 9, further comprising a step of rotating the elongate body prior to bending the elongate body.

20. The method of claim 9, wherein the step of inserting a first guidewire through the first lumen and out of the first exit opening to cannulate a pancreatic duct further comprises straightening the papilla.
